(19) United States
(12) Patent Application Publication
(10) Pub. No.: US 2023/0108122 A1
(43) Pub. Date: Apr. 6, 2023

(54) PULSED ELECTROMAGNETIC FIELD DEVICE WITH SUSTAINED MODULATION

(71) Applicant: Clineticz, LLC, Georgetown, TX (US)

(72) Inventors: George Michael Click, Georgetown, TX (US); Rick B. Yeager, Austin, TX (US); Dan A. Izzard, Georgetown, TX (US)

(21) Appl. No.: 17/909,367

(22) PCT Filed: Mar. 8, 2021
(86) PCT No.: PCT/US21/21295

§ 371 Date: Sep. 3, 2022

Related U.S. Application Data
(60) Provisional application No. 62/986,758, filed on Mar. 8, 2020.

(51) Int. Cl. A61N 2/02; A61N 2/00

(52) U.S. Cl. CPC A61N 2/02; A61N 2/008

(57) ABSTRACT

A PEMF device with a permanent magnet assembly with at least one permanent magnet delivers a strong and persistent magnetic field deep into tissue. A coil controller employs pulse width modulation and a phase controller to deliver a series of fast-rise-time current pulses to a coil assembly configured in proximity to the permanent magnet assembly. The current pulses generate magnetic filed flux and induce voltage by enhancing and retracting the deep magnetic field. The device appears to function as an antenna to transmit the coil electromagnetic field into adjacent tissue.

### RELATED APPLICATIONS

This is a US National Stage application of PCT application PCT/US21/21295, published as WO 2021/183410, which claims priority from U.S. Provisional Patent Application No. 62/986,758 filed on Mar. 8, 2020.

### FIELD OF INVENTION

This patent application relates to pulsed electromagnetic field (PEMF) devices. More particularly, the application relates to PEMF devices where magnetic flux is created by one or more coil positioned in proximity to at least one permanent magnet.

### BACKGROUND—PRIOR ART

Prior art PEMF devices generate electromagnetic field flux and induce voltage within the body by varying current through a coil. This non-invasive technique produces beneficial results as demonstrated in thousands of studies and research papers over more than five decades. The strength of the electromagnetic field can be increased by increasing the number of coil turns, by increasing current through a coil, or by increasing both the number of coils and the current. Magnetic field flux is designated as dB/dt where the “dB” represents the change in the magnetic field “B” for a time interval “dt”.

Low to medium intensity PEMF devices can produce a peak magnetic field up to about 200 gauss at high pulse frequencies directly from AC or battery power supplies. High intensity “impulse devices” such as transcranial magnetic stimulation (TMS) devices typically produce brief high amperage pulses at a peak intensity of 1,000-25,000+ Gauss, once or twice per second, by charging and discharging capacitors.

A 2003 NASA study reported that fast rise-time pulses promoted a 4× improvement for in vitro stem cell growth, but that sinusoidal pulses were not effective. U.S. Pat. No. 8,376,925 to Dennis et al describes commercial versions of high slew rate trapezoidal-wave pulsed electromagnetic field devices and the ICES-PEMF™, a low to medium intensity PEMF device.

U.S. Pat. No. 10,500,408 to Helekar et al. proposes advantages of a rotating permanent magnet device over conventional TMS devices. Strong permanent magnets can provide a strong magnetic field, but the flux created by rotating magnets is sinusoidal and less that effective than fast rise time coil pulses.

Most PEMF devices are operated at a frequency of 7-100 Hz. Some studies have reported that pathogen-specific “targeted frequencies” between about 1000 Hz and 2 MHz can kill or disable pathogens.

### SUMMARY

There are two fundamental limitations of coil-based PEMF devices to create effective magnetic field flux within tissue. First, the magnetic field strength drops sharply with distance from the coil with a decrease of 50% in the first 0.5 inch and over 99% in the first 3 inches. Second, it is not practical for an affordable device to create a strong magnetic field with pulse frequencies greater than a few pulses per second.

Applicants suggest that prior art PEMF devices cannot deliver combinations of pulse intensity and pulse frequency that effectively address many ailments. Low or medium intensity devices appear to have insufficient pulse intensity, and high intensity impulse devices appear to be have insufficient pulse frequency, to address many chronic conditions.

In one embodiment, a PERMAFLUX™ PEMF device has a flux module comprising a coil assembly having at least one coil configured in proximity to a permanent magnet assembly with at least one permanent magnet. The permanent magnets deliver a strong and persistent magnetic field deep into tissue. A coil controller employs pulse width modulation and a phase controller to deliver a series of fast-rise-time current pulses to a first coil to “ripple” the magnetic field.

The flux module can deliver a sustained modulation with duty cycles that are orders of magnitude higher than impulse PEMF devices, so PERMAFLUX can exceed the effective dosage of impulse devices by delivering many more pulses at a lower, and more benign, intensity. This ripple creates significant magnetic flux deep into the tissue without the need to recreate a strong magnetic field with each pulse. The pulse duration can be significantly longer than what is practical with many impulse devices.

When the first coil is energized with a positive current direction, the coil generates an electromagnetic field with the same polarity as the magnet assembly and thereby enhances the permanent magnetic field. When the first coil is energized with a negative current direction, the coil generates an electromagnetic field with the opposite polarity as the magnet assembly and thereby partially retracts the permanent magnetic field. A magnetic field flux is produced by the enhancement and retraction of the magnetic field. The device also appears to function as an antenna to transmit the coil electromagnetic field into adjacent tissue. Flux can be provided from a series of positive or negative pulses, an alternating series of positive and negative pulses, or other pulse patterns.

FIG. 8 is a front perspective view of an example AC powered PERMAFLUX device 100 showing an encapsulated flux module 500, a control box 340 with a lighted on/off switch 344, and cable 342 from the control box to the flux module 500. In this example, power is provided from an AC outlet and converted to 12 volts DC with a transformer (not shown). Portable devices will provide the flux module, controls, and battery or capacitor power supply within a hand-held housing.

FIG. 10 is an oscilloscope display showing an example 50 Hz coil signal with approximately 36% duty cycle. The lower trace shows a sequence 752 of nine sharp negative current pulses of about 300 microsecond duration which were generated by microprocessor pulse width modulation. The pulses have a rise time of less than 1 microsecond. The upper trace shows a subsequent sequence 750 of nine positive current pulses. In this example, a current of about 0.3 amps is directed to a 200-turn coil of 26 gauge copper wire wound around a permanent magnet assembly.

FIG. 11 shows an example upper trace analog display of the magnetic flux signal from a magnetic flux meter. The lower trace shows the positive current pulses 750, and the negative current pulses 752. This display is from a second oscilloscope and setup where the lower trace is displayed as inverted from FIG. 10. A magnetic field flux pulse is generated by each current pulse. The positive fluxes 760 created by the positive current pulses 750 are offset from the negative fluxes 762 created by the positive current pulses 752. The flux pulses have a duration of approximately 1 millisecond and induce voltage in the brain or tissue.

FIGS. 12A-12D illustrate example pulse rates and relative intensities for prior art PEMF devices and PERMAFLUX over a one second time frame. For illustration, all devices are shown at the same 50 Hz frame intervals 1-50.

FIG. 12A is an example sinusoidal resonant PEMF device with 50 sinusoidal pulses 770a, 770b, 770c, 770d . . . per second at a peak intensity of about 200 gauss produced in each cycle. The slow change in coil current creates longer pulse rise times, resulting in a smaller dB/dt than the other devices.

FIG. 12B is an example PEMF device where sharp positive 772a, 772b, . . . or negative 773a, 773b, . . . current pulses of about 200 microsecond duration is delivered in each of the 50 cycles with a peak intensity of about 200 gauss produced in each cycle. Most of these devices are operated at a lower intensity. The magnetic field flux produced by this pattern is substantially stronger than in the sinusoidal devices because of the fast pulse rise times. The pulses are shown as vertical lines, but are actually trapezoidal in shape with steep rise and fall times.

FIG. 12C is an example of impulse PEMF devices where a single pulse with 3 nanosecond to 400 microsecond duration is delivered once per second. The example shows a single pulse 774a in the first frame and no activity for frames 2-50. The magnetic field flux produced by this pattern is likely to be the strongest of the examples, if very short nanosecond pulses are not too fast to create effective flux, but the pulse rate is the slowest of the examples.

High intensity appears to be important, but not sufficient, to treat chronic ailments. If medium intensity resonant devices could be effective, then they should have been well-established in the last 15 years. Likewise, if impulse devices were effective, then more successes should have been documented in the last 10 years. Applicants suggest that both types of devices can offer some benefit, but that each has a fundamental limitation.

FIG. 12D is an example of PERMAFLUX operating at 50 Hz with nine negative current pulses 752a, 752b, 752c, 752d, . . . and nine positive current pulses 750a, 750b, 750c, 750d, . . . in each of the fifty 0.020 second cycles. This example delivers the highest pulse rate of 900 pulses per second, with each pulse having a fast rise time of less than 2 microseconds, and about 300 microsecond duration. In addition to the faster pulse rate, the magnetic field flux has a substantially deeper reach into tissue than a device with an equivalent coil size and current.

### REFERENCES

[1] The ARRL Antenna Book for Radio Communications, 24th Edition, Published by (ARRL) The American Radio Relay League, Inc.
[2] The ARRL Handbook for Radio Communications, (Six Volume Set), Ninety-Eighth Edition; First Printing (2020) Published by (ARRL) The American Radio Relay League, Inc.
[3] https://www.kjmagnetics.com/calculator.asp

### BRIEF DESCRIPTION OF FIGURES

FIG. 1 is a cross section view of an example device flux module.

FIG. 2 is a front view of the example flux module of FIG. 1.

FIG. 3 is a front view of a example magnet assembly with a plurality of cylindrical magnets.

FIG. 4 is a front perspective view of another magnet assembly.

FIG. 5 is an example of a flux module with two coils.

FIG. 6A is a cross section view of another example flux module.

FIG. 6B is a cross section view of another example flux module.

FIG. 6C is a cross section view of another example flux module.

FIG. 6D is a cross section view of another example flux module.

FIG. 6E is a cross section view of another example flux module.

FIG. 7 is an example pulse control schematic.

FIG. 8 is a front perspective view of a first embodiment device.

FIG. 9 is a top perspective view of an example magnet support.

FIG. 10 is an oscilloscope display below showing an example 50 Hz coil signal.

FIG. 11 is an oscilloscope display of magnetic flux pulses and coil pulses.

FIG. 12A (PRIOR ART) is an example 50 Hz sinusoidal PEMF device coil signal.

FIG. 12B (PRIOR ART) is an example a 50 Hz sharp pulse PEMF device coil signal.

FIG. 12C (PRIOR ART) is an example impulse PEMF with a 1 Hz coil signal.

FIG. 12D is an example embodiment 50 Hz coil signal with nine negative current pulses and nine positive current pulses each cycle.

### DESCRIPTION

In this specification, the term “PEMF” means pulsed electromagnetic field. The term “PEMF therapy” refers to the use of a PEMF device on a living organism, including the human body; dogs, horses, and other animals; or plants. The embodiments described herein can be considered PEMF devices where one or more coil supplies a pulsed electromagnetic field by “rippling” a permanent magnetic field, or PEMF devices where the combination of a permanent magnet assembly and a coil assembly acts as an antenna to broadcast the electromagnetic fields created by the coil and magnet assemblies.

The term “slew rate” refers to the calculated magnetic flux value “dB/dt” where dB is the change in magnetic field strength B, and “dt” is the change in time. Some PEMF devices with an ultra-fast pulse rise time can yield a high calculated dB/dt, but might have little practical benefit because of the small actual magnetic field intensity.

The term “coil controller” refers to the conversion of a power supply to a series of discrete current pulses to a coil. In some examples, the coil controller may be adjustable to produce various pulse intensities, pulse frequencies, and pulse durations.

“Pulse width modulation” is a method of controlling current to a device by producing trapezoidal waves, where input DC voltage is switched “on” and “off” to create nominal square waves. In this specification, the terms “current control” and “current controller” are used in preference to the terms “voltage control” or “voltage control”.

The term “North polarity” means that a magnet assembly has magnet(s) with a North pole oriented toward a subject. The term “positive current” means the current direction through a coil which produces an electromagnetic field with North polarity oriented toward a subject.

Example embodiments describe a pulsed electromagnetic field device configured to apply a fluctuating electromagnetic field to a subject organism. The devices have at least one flux module comprising a permanent magnet assembly with at least one permanent magnet, and a coil assembly comprising at least one first configured in proximity to the permanent magnet assembly. A first coil controller is configured to intermittently apply current from a power supply to a first coil.

Some embodiments have a plurality of parallel cylindrical magnets where the first coil is wound around the permanent magnet assembly and configured perpendicular to the longitudinal axes of the magnets.

The devices are held or supported near or against a subject organism, such as a human, animal, or plant. In one example, the pulse controller delivers a plurality of positive and negative current pulses with rise times less than 5 microseconds to generate magnetic filed flux and to induce voltage within the organism.

### Example—Flux Module with Multiple Cylindrical Magnets and Single Coil

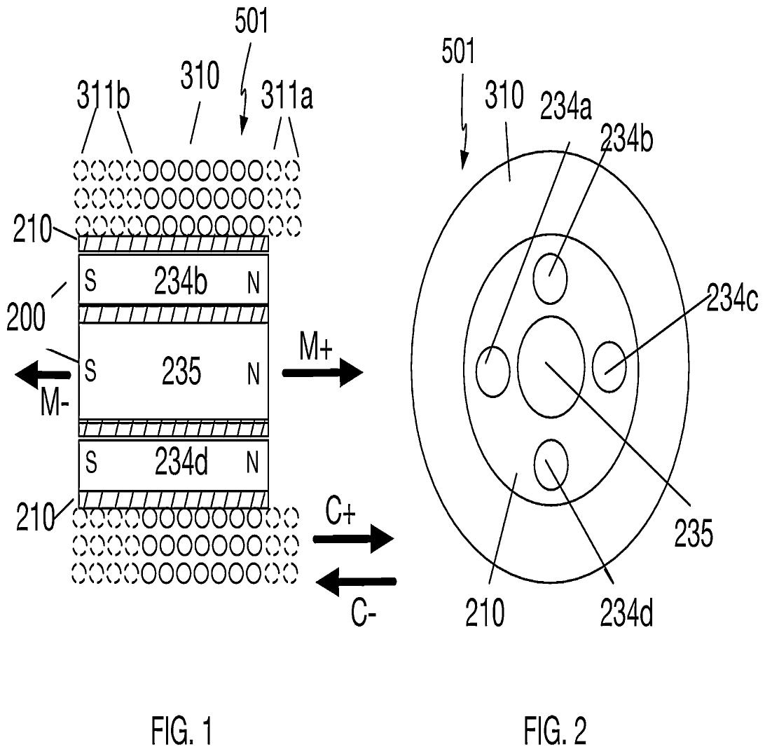

FIGS. 1 and 2 are a cross section and front view of an example flux module 501. In this example, a single coil 310 is provided in proximity to a magnet assembly 200. The coil is wound around the magnet assembly housing 210, which also serves as a spool for the coil winding. The magnet assembly comprises five cylindrical N52 NIB permanent magnets, which are arranged symmetrically with smaller magnets 235a, 235b, 235c, and 235d oriented concentric to a center magnet 234 which has a larger diameter than the other magnets. In one example, the center magnet has a 0.5 inch diameter and a length of 1.25 inches, and the outer magnets have a 0.25 inch diameter and a length of 1.25 inches.

The single coil 310 is wound symmetrically about an axis parallel to the longitudinal axis of the magnet assembly, and extends around a substantial length of the magnet assembly. In other examples, the coil orientation may be asymmetric to the magnet assembly, the coil may be the same width as, or shorter than, the magnet assembly, or overhang the magnet assembly in one or both directions as indicated by coil sections 311a and 311b. The coil width to diameter aspect ratio may be substantially more or less than shown.

The permanent magnet assembly provides a permanent magnetic field M+ in the direction of tissue (not shown). In this specification, M+ is designated as being directed from the North pole of the magnet assembly. In some configurations, the coil leads (not shown) are introduced at the South pole end of the device. In other configurations, the coil leads are introduced in a manner that does not obstruct either pole region of the magnet assembly, and the device may be used with either the North polarity M+, or the South polarity M−, field lines directed toward tissue. The latter configuration permits the spaced apart placement of two or more devices in various opposing or attracting polarity orientations as discussed below.

In one example, the coil has approximately 200 turns of 26 gauge insulated wire. In other examples, the number of turns may be less, or substantially higher, and various wire size may be used. This example coil configuration appears to be reasonably “tuned” for various magnet assembly configurations, but is likely to be optimized with further modeling and empirical testing. At least two factors appear to influence coil design. First, there are traditional tradeoffs in all coil-based devices between coil intensity from more coil turns and/or higher coil current, versus the duty cycle and heat buildup in the coil due to wire resistance. Second, the combination of magnet and coils creates an antenna which can be “tuned” to reduce reflected signal and thereby increasing the transmission of coil signal. The reflected signal from inefficient tuning generates heat in the coil control, and increases the energy demand of the device.

The flux module acts as an antenna to transmit the coil electromagnetic field. In order to reduce feedback and increase transmission, it is desirable to “tune” the system as described in references [1, 2]. When the assembly is tuned, less power is wasted on feedback to the controller, and the coil may be energized with less current. In prototype devices, tuning reduced the current from a 12 volt power supply from about 1 amp to less than 0.3 amps, and eliminated heat buildup in the controller. A coil of approximately 200 turns appears to perform well with many N52 NIB permanent magnet assemblies, but is likely to be further optimized.

Since antenna signals can be focused or directed with parabolic or other shaped reflectors, it should be possible to provide rear and/or peripheral shielding with various materials to improve the electromagnetic field transmission. In one example, shielding or reflector elements may be configured as a parabolic antenna positioned behind or around the flux module.

Testing indicates that example assemblies are tuned to provide sufficient intensity and depth of field for good therapeutic results for chronic conditions without cooling of either the coil or the control. Further clinical testing may suggest a need for greater coil intensities to address various conditions, and coil or cooling can be provided with active cooling or static cooling, such as cooling rods.

From one perspective, devices appear to work as an antenna which delivers the coil magnetic field flux substantially deeper into tissue than can be achieved with the coil only.

From another perspective, as suggested by the observed oscillation of small permanent magnets suspended along return magnetic field lines of force, the device appears to “ripple” the permanent magnetic field lines by the coil alternately partially repelling the magnet field lines in the direction of C1 when coil and magnet polarity is matched, and partially attracting the magnet field lines in the direction of C2 when the coil and magnet polarity is opposed. This perspective suggests that the coil would be effective with a sequence of only positive or negative current pulses.

It is not practical in medium intensity PEMF devices to provide a large enough coil or enough current to produce a constant magnetic field to match the strength of a strong permanent magnet field. Likewise, it is not practical to create pulses of that strength without accumulating energy to discharge into stronger pulses. Impulse devices use capacitors to provide short pulses that can match or exceed the permanent magnet field, but practical devices cannot be recharged to peak intensity at a rate much greater than a few pulses per second.

Various embodiments of flux modules deliver an increased magnetic flux deeper into tissue that prior art medium intensity PEMF devices. Embodiments have been successfully tested at 900 pulses per second without the need for supplemental coil or controller heat removal, and higher frequencies are likely to be feasible with additional optimization such as faster pulse rise times, shorter pulses, or heat removal.

High intensity impulse PEMF devices can be adapted to incorporate permanent magnet assemblies in order to deliver greater magnetic field flux at higher pulse frequencies (and therefore shorter treatment session times) than can be provided by coils alone.

To date, prototype devices have used the strong N52 NIB magnets, approximately 200 turn coils, and a coil current that can be applied to achieve relatively long pulse duration and relatively high pulse frequency without producing significant heat build-up. Prototype devices have shown unexpected beneficial results for a number of chronic conditions, so development has been directed to improving pulse control, reducing pulse rise times, and designing portable devices. Future research will test various other magnet and coil sizes and configurations, conduct finite element analysis and otherwise characterize and optimize design and control parameters. It may be also be desirable, in some cases, to use stronger permanent magnets as they become available, or weaker permanent magnets.

### Example Configurations

In other examples, one or more magnets may be used in the magnet assembly, the magnets may have the same dimensions and strengths, different dimensions, different strengths, and symmetric or asymmetric configurations relative to circular or other layouts. In other examples, the magnet assembly comprises one or more permanent magnet of various shapes and sizes in symmetric or asymmetric alignment, and similar or various magnetic strengths. These examples describe various sizes and orientations of cylindrical or ring magnets. Other magnet shapes, such as bar magnets may be used to achieve different magnetic field characteristics. Magnets within a magnet assembly typically have the same polarity alignment. Other polarity alignments could be used to modify magnetic field profiles.

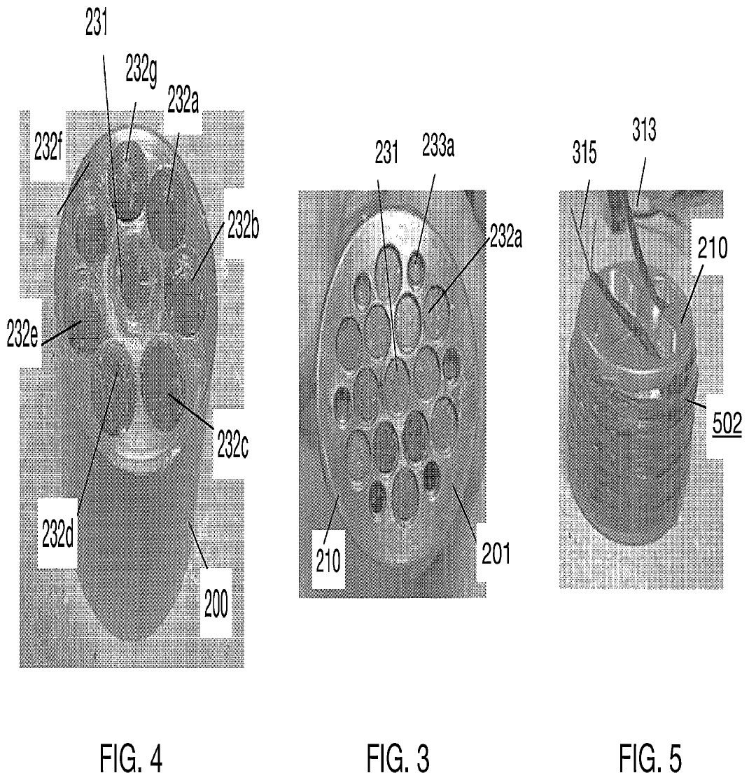

FIG. 3 is a front view of a magnet housing 210 with a central 0.5 inch diameter cylindrical magnet 231, twelve other 0.5 inch diameter cylindrical magnets 232; and six 0.25 inch diameter cylindrical magnets 233.

FIG. 4 is a front perspective view of a magnet assembly 200 with a central 0.5 inch diameter cylindrical magnet 231 and six other 0.5 inch diameter cylindrical magnets 232a-232g.

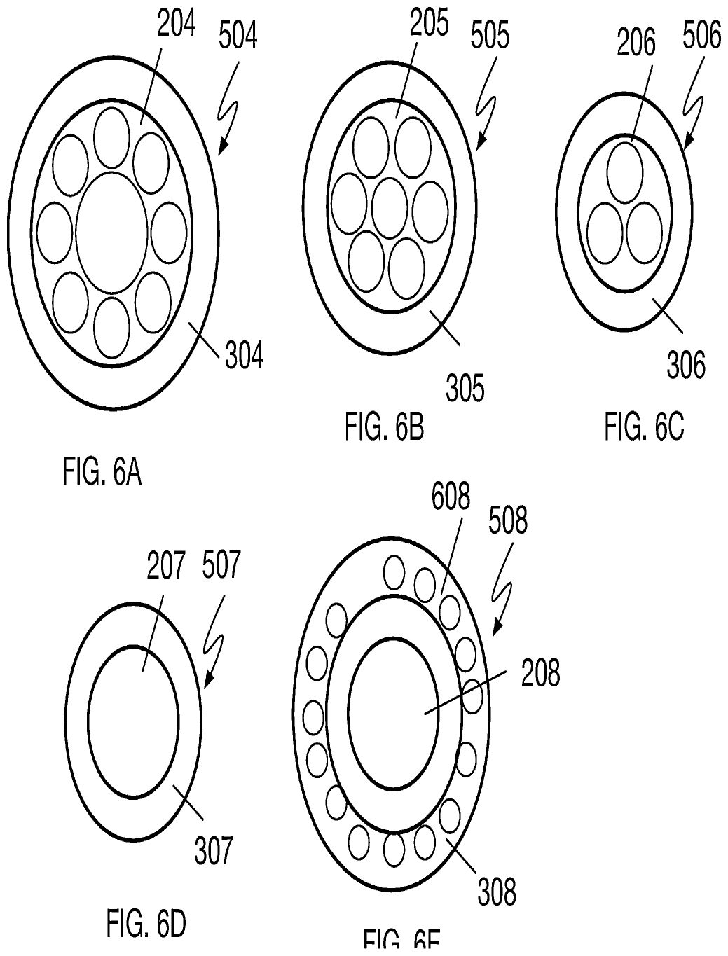

FIG. 6A is a cross section view of an example flux module 504 with a coil assembly 304 and a magnet assembly 204 having a central magnet that is larger than eight surrounding magnets. An example magnet module with a 0.25 inch central magnet and 0.125 inch smaller magnets provides good magnetic field depth with a more focused treatment area.

FIG. 6B is a cross section view of an example flux module 505 with a coil assembly 305 and a magnet assembly 205 with a central magnet surrounded by six magnets of the same size.

FIG. 6C is a cross section view of an example flux module 506 with a coil assembly 306 and a magnet assembly 206 with three magnets of the same size.

FIG. 6D is a cross section view of an example flux module 507 with a coil assembly 307 and a magnet assembly 207 having a single central magnet.

FIG. 6E is a cross section view of an example flux module 508 with a coil assembly 308, a first magnet assembly 208, and a second magnet assembly 608. In this example, the first magnet assembly has a single central magnet, and a second magnet assembly 608 is configured over the coil assembly with a plurality of asymmetrically-arranged smaller magnets. In contrast to the focus provided by the latter example in FIG. 6A, this type of arrangement expands the area of the magnetic field.

In other examples, the magnet assembly comprises a central cylindrical ring magnet oriented within the hole of a ring magnet.

TABLE 1
Field Strength (mT) − 1

| Distance | | | | | | | | |
|---|---|---|---|---|---|---|---|---|
| from Axis | Distance from face (cm) | | | | | | | |
| (cm) | 1.0 | 2.0 | 3.0 | 4.0 | 5.0 | 6.0 | 7.0 | 8.0 |
| 3.0 | 49 | 20 | 13 | 11 | 8 | 6 | 5 | 3 |
| 2.0 | 81 | 49 | 26 | 17 | 11 | 7 | 5 | 4 |
| 1.0 | 135 | 65 | 33 | 22 | 13 | 9 | 6 | 4 |
| 0.0 | 155 | 76 | 46 | 23 | 14 | 9 | 6 | 4 |
| 1.0 | 147 | 67 | 37 | 22 | 14 | 8 | 6 | 4 |
| 2.0 | 96 | 50 | 30 | 18 | 13 | 7 | 5 | 4 |
| 3.0 | 41 | 28 | 17 | 14 | 9 | 6 | 4 | 3 |

A magnetic strength calculator [REF 3] demonstrates the effect of magnet shape and dimensions on peak intensity and intensity at distance from the head. Without being limited by theory, applicants suggest that these calculated differences are due, in part, to the relative number of “shortcut” magnetic field lines of force between the poles. This suggests that magnetic field intensity at a distance might be increased with various arrangements of multiple smaller magnets rather than a single larger diameter magnet. The use of smaller magnets also reduces device weight and cost.

FIG. 5 is an example of a flux module 502 with first coil lead 313 and second coil lead 315. Table 1 shows measured magnetic field strengths versus x and y position for the flux module 502. The magnetic field strength decreases approximately with the square of the distance from the magnet.

### Method of Manufacture

FIG. 9 is a top perspective view of an example aluminum magnet support 210 with a plurality of magnet cavities 212 and a coil retention rim 214. In other examples, the support is produced by urethane casting. One or more coil is wound around the spool housing and magnets are secured in the cavities. Leads are secured to the coil(s) and the magnet and coil assembly is encapsulated in a fast-curing urethane pour mold with a handle portion to facilitate gripping the flux module. The coil leads are connected or soldered to a coil controller circuit board secured in a housing. The housing supports a lighted on/off switch and a power supply adapter port. In portable devices, the magnet and coil assembly, coil controller circuit board, and battery or capacitor power can be provided in a single housing.

### Coil Control

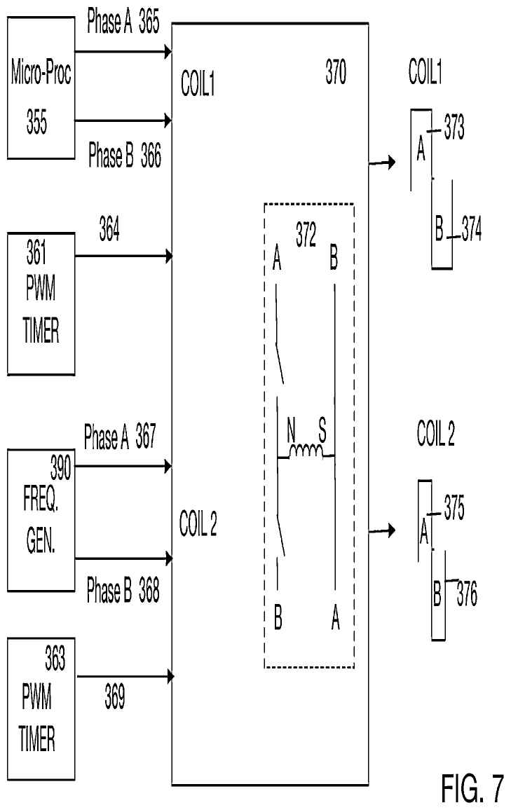

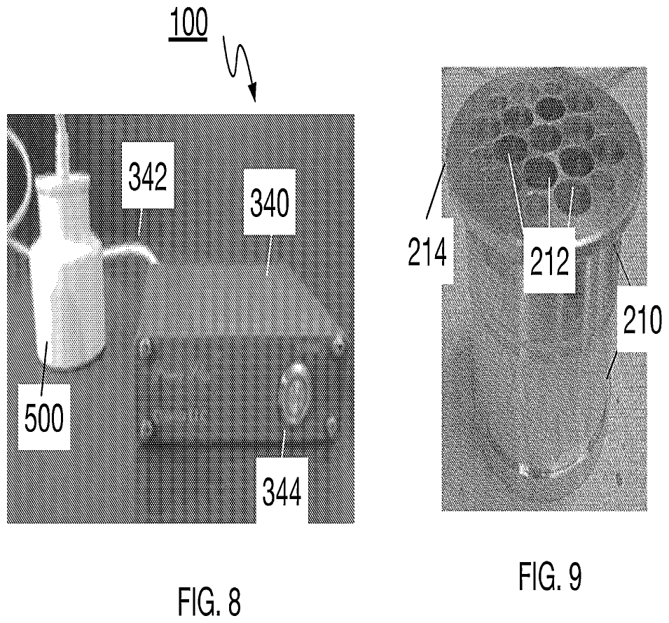

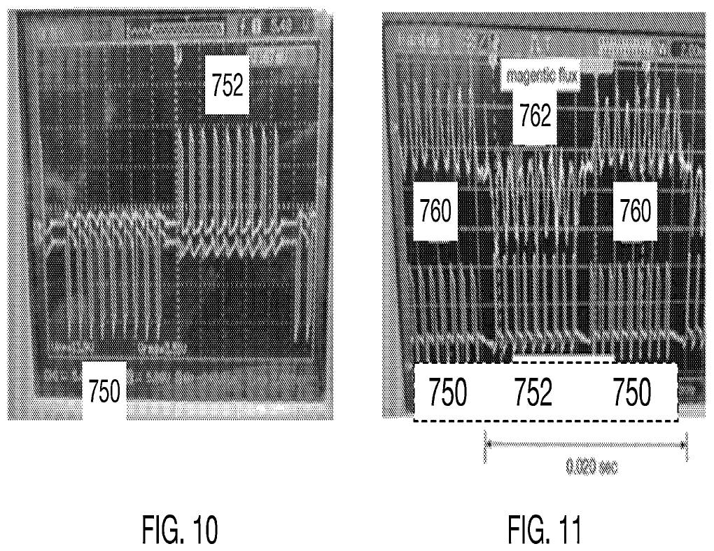

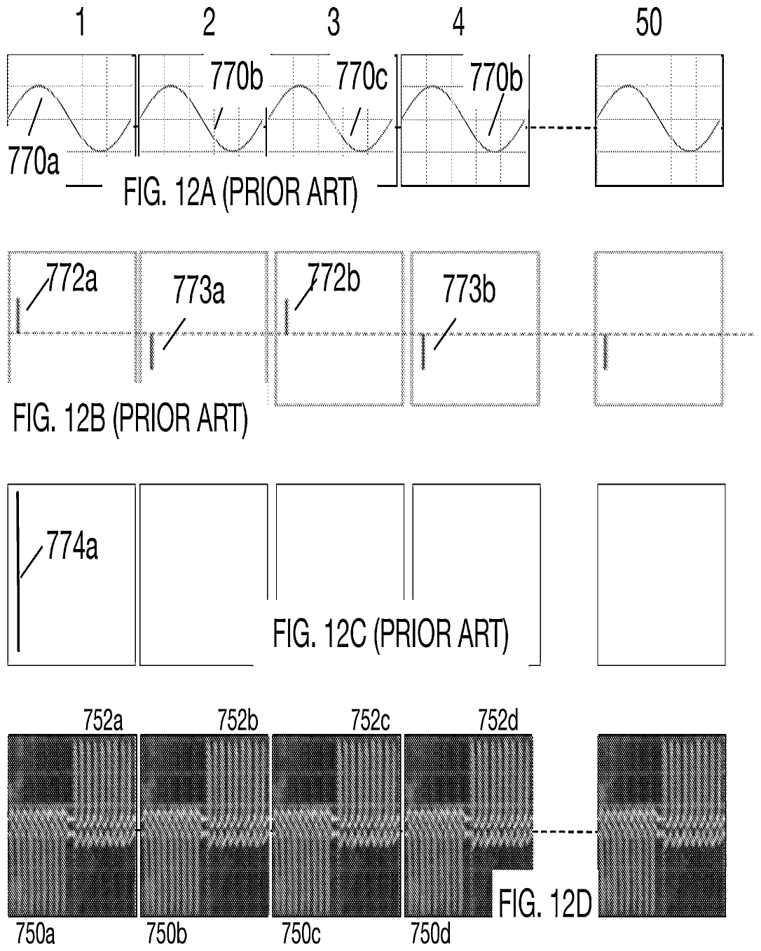

FIG. 7 is an example pulse control schematic. A microprocessor 355 and a pulse width modulation timer 361 provide a first coil signal to a phase controller 370. The phase controller has an H-bridge 372 that creates positive current pulses 373 and negative current pulses 374. In various examples, a second target frequency coil may be driven by a constant frequency or with a variable frequency such as range of audio frequencies. FIG. 7 shows a second coil signal created by a frequency generator 390 and a pulse width modulation timer 361. A phase controller generates positive current pulses 375 and negative current pulses 376.

Controls may be provided to select pre-programmed pulse protocols, or to select one or more of pulse intensity, pulse frequency, pulse duration, pulse pattern, or session timing. Prior art has suggested that organisms may adapt to constant frequencies, and that it may be desirable to randomly or otherwise vary the coil frequency and/or coil intensity. For reasons described below, prototype devices were simplified.

### Device Performance

Measurements of induced voltage from flux modules are substantially greater than the induced voltage from the coil only. In one example, the difference ranged from about 40% at close range to about 25% at distances of a few centimeters.

Over 30 prototype Permaflux devices have been built with various N52 NIB magnet sizes and configurations, and approximately 200 turn coils with 0.3 amp pulse width modified current at 50 Hz with 9 positive and 9 negative current fast-rise pulses per cycle. The device is typically held against or near the skin or head for two minutes per site. Significant beneficial results have been reported for both in-clinic and at-home device use for a number of chronic conditions including pain and inflammation.

Devices used in two minute sessions appear to provide more effective symptom and/or substantive relief than 20-30 minute TMS treatments for a variety of chronic conditions. These results suggest the importance of an overall “dosage” parameter that incorporates the number of pulse events, the intensity of the events, and the effective reach of those events relative to a target region in the body or brain.

### Applicators and Portable Devices

In another embodiment, a plurality of flux modules are provided in order to induce voltages over a larger area or volume. As examples, a plurality of flux modules may be affixed to a helmet, headband, belt, vest, or sling support; or provided in a paddle, mat, or mattress housing to deliver flux over a larger area. While magnets having a length to diameter aspect ratio of 2.5 to 5 or more appear to be desirable to improve magnetic field intensity at increased distances from the flux module, larger applicators can employ lower aspect ratio magnets.

The AC powered examples described above have a coil control circuit board that is small enough to be incorporated in a hand-held portable device. In one example, three lithium ion rechargeable batteries, can provide a total voltage of over 11 amps with a battery life that can support many treatment sessions before recharge.

Pods are portable flux modules incorporating smaller power supplies that can be quickly recharged with direct or induction recharged for a few treatments.

### Remote Therapy

Prototype devices were designed, at clinic request, for simplicity of operation, with only an on/off switch. Therapy sessions were timed, and the device could be either held against the subject for maximum intensity, or could be spaced away from the subject by up to a centimeter to reduce intensity by about half.

There are several advantages to this approach relative to more complex programming or control options, including consistency of operation, ease of administration or supervision, and lower patient intimidation.

In one example of a home use treatment protocol for a particular ailment, a medical provider can specify device location, device contact or spacing, and treatment session times and frequency. Since the device price is very low compared to clinical devices, the device can be sold, rented, or borrowed for home use; and home use sessions can be monitored by medical staff by a simple video link such as ZOOM™ or FACETIME™.

By reducing the need for study participants to travel to a supervised test site, video monitoring can also dramatically reduce administration costs and increase feasible participant sample size for controlled studies of device effectiveness for various ailments.

In other examples, the device may include a communications link which permits remote programming of pulse control parameters and session timing.

### Target Frequencies

Operating devices at 900 pulses per second appears to be particularly effective at addressing pain and chronic inflammation, and in strengthening immune response. Other higher or lower pulse rates are also likely to be effective.

In other examples, a second coil is provided, and is operated at a higher targeted frequency than the first coil. In one example, the second coil can be operated a constant frequency in the range of 2000-5000 Hz. Literature suggests that frequencies as high as 1.6 megaHz may be effective. A dual coil device provides two base frequencies as well as sums and differences of the base frequency harmonics. In other examples, the second coil can be operated a variable frequencies. Limited in vitro testing suggests the dual coil device operating at a specific target frequency may be effective in killing or weakening pathogens.

Dual Heads with Attractive or Repulsive Orientations

Despite the significant drop in field intensity with distance from the head, the testing of single devices has shown effectiveness for a wide range of ailments.

In some cases, it may be desirable to provide a more intense and focused magnetic field over a longer distance. The potential effectiveness of using spaced-apart dual flux modules with attracting or opposing heads can be demonstrated with a chamber having iron filings suspended in a viscous liquid. When a first flux module is placed on opposite sides of a chamber several inches from a second flux module having opposite polarity, a horizontal column of filings is suspended between the modules—indicating a strong and near-uniform magnetic field. This type of dual device placement can provide a strong and focused field across a head, torso, leg, or arm.

In other cases, two or more same-polarity devices may be positioned to create repulsive interference regions. Various devices can be positioned to generate a desired magnetic field interference boundary location and shape; and to move that boundary back and forth relative to a desired region of interest.

These types of opposed or attractive polarity configurations may also serve as antennae when a coil is provided with at least one of the magnet assemblies.

### Impulse Devices

The flux module approach shifts the intensity versus distance curve for low to medium intensity PEMF devices toward greater intensity at a given distance from the coil. Since these devices can operate at any pulse frequency, the primary benefit is in improving effective magnetic flux at distance from head.

In some examples, it may be desirable to provide flux modules with much higher coil current than can be supplied without a voltage multiplier. Prior art impulse devices can only provide peak intensity at low pulse rates. For example, an impulse PEMF vendor reported 2020 data showing a peak intensity of 1400 Gauss at 0.54 pulses per second; but dropping to 350 Gauss at 6 pulses per second and to 220 Gauss at 10 pulses per second.

The flux module approach of combining a magnet assembly with high intensity coils in impulse PEMF devices can provide greater intensity, more frequent pulses, or both.

For a given intensity at or below peak, a flux module approach could enable a higher pulse rate, so that treatment times could be reduced by a factor of 2-3× or more. In other cases, such as pelvic muscle contraction, TMS, or migraine therapy, where slow pulse rates may be preferred, a flux module approach —particularly in a dual head opposed polarity configuration—could substantially reduce the required coil size or coil current to match or exceed coil-only performance.

### Ailments

Based on initial clinic use, home use, and device measurements, the embodiments described herein appear to be more effective than prior art PEMF devices in addressing a variety of acute and chronic ailments. The devices appear to be particularly effective in reducing inflammation so that the body's immune system can address those ailments. Many of the applications of PEMF are summarized in U.S. Pat. No. 8,376,925 to Dennis et al. and that list is substantially repeated in the attached claims.

While these embodiments have been described with emphasis on the embodiments, it should be understood that within the scope of the appended claims, the embodiments might be practiced other than as specifically described herein. In particular various configurations of coil assemblies, magnet assemblies, and pulse intensities, pulse duration, pulse rise times, treatment protocols, and device placement may be used.

What is claimed is:

1. A pulsed electromagnetic field device configured to apply a fluctuating electromagnetic field to a subject organism, the pulsed electromagnetic field device comprising
a flux module comprising
a permanent magnet assembly comprising
a first permanent magnet having a North polarity region, and a South polarity region, such that the North polarity region and the South polarity region are spaced apart along a first permanent magnet axis, the first permanent magnet being oriented such that when the flux module is is positioned in proximity to a subject organism, the first permanent magnet projects a permanent magnetic field into the subject organism such that the permanent magnet assembly has a substantially solid cross section perpendicular to the first permanent magnet axis, and
a coil assembly configured in proximity to the permanent magnet assembly, the coil assembly comprising a first coil wound about the permanent magnet assembly;
a power supply; and
a first coil controller configured to ripple the magnetic field by intermittently applying current from the power supply to the first coil, such that
when no current is applied to the first coil, the permanent magnetic field is not altered, and
when current is applied to the first coil, the first coil generates a secondary magnetic field that alters the permanent magnetic field.

2. The pulsed electromagnetic field device of claim 1 wherein the permanent magnet assembly comprises a plurality of parallel cylindrical magnets, each magnet having a first end with North polarity and a second end with South polarity, and a longitudinal axis between the first end and the second end; and the first coil is wound around the permanent magnet assembly with respect to the first permanent magnet axis, and configured perpendicular to the longitudinal axes of the cylindrical magnets.

3. (canceled)

4. The pulsed electromagnetic field device of claim 1 wherein the permanent magnet assembly further comprises a second permanent magnet having a North polarity region and a South polarity region spaced apart along a second permanent magnet axis that is the same as, or parallel to, the first permanent axis.

5. The pulsed electromagnetic field device of claim 2 wherein each cylindrical permanent magnet has a magnetic field strength equivalent to at least an N52 NIB magnet.

6. The pulsed electromagnetic field device of claim 1 further comprising a permanent magnet housing, such that the first coil is wound around the permanent magnet housing.

7. The pulsed electromagnetic field device of claim 1 wherein the first coil has between 100 and 500 turns.

8. The pulsed electromagnetic field device of claim 1 wherein the power supply comprises line voltage, at least one battery, or at least one capacitor.

9. The pulsed electromagnetic field device of claim 8 further comprising a transformer configured to convert AC line voltage to 12 volt DC.

10. The pulsed electromagnetic field device of claim 1 wherein the first coil controller is further configured to provide pulse width modulation.

11. The pulsed electromagnetic field device of claim 1 wherein the first coil controller is further configured to provide a sequence of positive and negative current pulses from an input voltage of 6 volts to 24 volts, such that each pulse has a rise time less than 5 microseconds.

12. The pulsed electromagnetic field device of claim 1 wherein the first coil controller is configured to deliver a sequence of current pulses in the range of 1 to 1000 pulses per second.

13. The pulsed electromagnetic field device of claim 1 wherein the first coil controller further comprises a voltage multiplier.

14. The pulsed electromagnetic field device of claim 1 further comprising a voltage multiplier configured to deliver a series of current pulses sufficient to produce a peak magnetic field intensity in the range of 1,000 to 25,000 gauss.

15. The pulsed electromagnetic field device of claim 1 wherein the coil assembly further comprises a second coil wound about the permanent magnet assembly, and a second coil controller.

16. The pulsed electromagnetic field device of claim 15 wherein the second coil controller is configured to deliver at least 1000 current pulses per second.

17. The pulsed electromagnetic field device of claim 1 further comprising a focus element positioned around at least a portion of the coil assembly.

18. The pulsed electromagnetic field device of claim 17 wherein the focus element is a parabolic antenna.

19. The pulsed electromagnetic field device of claim 1 wherein the permanent magnet assembly comprises a plurality of cylindrical permanent magnets, each magnet having a length of at least one inch; the first coil has 150-250 turns; and the first coil controller is configured to deliver a sequence of positive and negative current pulses, from an input voltage of 6-14 volts, at a rate of 800 to 1000 pulses per second, where each pulse has a rise time less than 5 microseconds.

20. (canceled)

21. A method of creating a magnetic field flux within a subject organism, the method comprising providing a first pulsed electromagnetic field device comprising
a first flux module comprising
a permanent magnet assembly comprising
a first permanent magnet having a North polarity region, and a South polarity region, such that the North polarity region and the South polarity region are spaced apart along a first permanent magnet axis, the first permanent magnet being oriented such that when the flux module is is positioned in proximity to a subject organism, the first permanent magnet projects a permanent magnetic field into the subject organism, such that the permanent magnet assembly has, a substantially solid cross section perpendicular to the first permanent magnet axis, and
a coil assembly configured in proximity to the permanent magnet assembly, the coil assembly comprising a first coil wound about the permanent magnet assembly;
a power supply; and
a first coil controller configured to ripple the magnetic field by intermittently applying current from the power supply to the first coil, such that
when no current is applied to the first coil, the permanent magnetic field is not altered, and
when current is applied to the first coil, the first coil generates a secondary magnetic field that alters the permanent magnetic field.
positioning the first flux module in proximity to a subject; and
delivering a plurality of current pulses from the first coil controller to the first coil, thereby rippling the permanent magnetic field in order to create creating a magnetic field flux within the subject organism.

22. The method of claim 21 further comprising remotely supervising the positioning the first flux module and delivery of the plurality of current pulses

23. (canceled)

24. (canceled)

25. The method of claim 20 further comprising
positioning the first pulsed electromagnetic field device near a subject organism with the North polarity region of the first permanent magnet oriented toward the subject organism; and positioning a second permanent magnet assembly or a second pulsed electromagnetic field device near the subject organism, spaced apart from the first pulsed electromagnetic field device, with a South polarity region or a North polarity region of a permanent magnet assembly oriented toward the subject organism.

26. (canceled)

27. (canceled)

28. (canceled)

29. (canceled)

30. The method of claim 20 wherein the subject organism is a human with an ailment selected from the group consisting of:
a cellular dysfunction, an extracellular matrix disruption, focal or general sarcoidosis, an allergy or hypersensitivity of skin, an allergy or hypersensitivity of mucous membranes, an allergy or hypersensitivity of a pulmonary system, a chronic open wound, an ulceration of skin, a pressure ulcer, a decubitus ulcer, a damaging effect of ionizing radiation, a damaging effect of chemotherapy, urinary or fecal incontinence related to damaged nerves and/or muscles of a urogenital system, a presence of bacteria, a presence of a virus, a presence of prions, pain, acute or chronic inflammation, acute or chronic swelling, edema, an inflammatory response, acute inflammation following injury or trauma, a chronic or acute condition related to inflammation or edema, fibrosis, necrosis, inflammation and associated tissue disruption arising from autoimmune reactions or autoimmune hyperactivity, a presence of pathogens or foreign bodies, inflammation of joints, articulations of a spinal column, articulations in a spinal system, swelling of a spinal cord resulting from injuries, swelling of the spinal cord resulting from destructive plaques, inflammation around nerves, and combinations thereof;
a. the ailment is a degenerative condition associated with aging and inflammation selected from the group consisting of: degenerative joint disease, arthritis, inflammatory arthritis, palindromic rheumatism, non-infectious arthritis, infectious arthritis, joint damage, vasculitis, phlebitis, arteritis, lymphangitis, rheumatism, fibromyalgia syndrome, non-articular rheumatism, regional pain syndrome, sarcopenia, chronic low-grade inflammation, calcium deposits, and combinations thereof;
b. the ailment is an acute or chronic inflammatory response and/or a subsequent disease state of a cardiovascular system selected from the group consisting of: vascularitis, endocarditis, atherosclerosis, coronary heart disease, stroke, peripheral artery occlusive disease, pericarditis, and combinations thereof;
c. the ailment is an inflammatory bowel disease selected from the group consisting of: Crohn's disease, chronic prostatitis, inflammation due to hypersensitivities, inflammatory bowel disease, endometriosis, chronic pelvic pain, cysts, abscesses, arthritis, calcium deposits, hernias, and combinations thereof;
d. the ailment is a disease secondary to a pathological acute or chronic inflammatory response selected from the group consisting of: a neurodegenerative disease of a central nervous system, Alzheimer's, dementia, a neurodegenerative disease of a peripheral nervous system, transverse myelitis, a neuroinflammatory condition, and combinations thereof, wherein the neuroinflammatory condition is phantom limb pain, neuropathic pain, nociceptive pain, chronic pain, or chronic idiopathic pain;
f. the ailment is an idiopathic inflammatory demyelinating disease selected from the group consisting of: a neuropathy resulting from Guillain-Barre syndrome, lupoid hepatitis, mixed connective tissue disease, mixed connective tissue disease, Sharp's syndrome, Meniere's disease, multiple sclerosis, myasthenia gravis, myositis, myalgia, and combinations thereof;
g. the ailment is acute inflammation caused by or relate to a member of the group consisting of: frostbite, chilblains, pernio, acral ulcers, acrocyanosis, psoriasis, trench foot, a reactive neutrophilic cutaneous condition, recalcitrant palmoplantar eruptions, heat edema, heat rash, Miliaria rubra, sunburn, jogger's nipple, edema, cutaneous edema, contact edema, lymphedema, derangement of control of a volume of interstitial fluid, compartment syndrome, mechanical or chemical trauma to the tissue, ulcerative inflammation, regrowth of hair erectile dysfunction, and combinations thereof;
h. the ailment is an underlying chronic inflammatory response mechanism or a lingering symptom associated with an inflammatory disorder selected from the group consisting of: edema, cutaneous edema, contact edema, lymphedema, derangement of control of the volume of interstitial fluid, compartment syndrome, hand-arm vibration syndrome, vibration white finger, temporomandibular joint disorder, conditions of subcutaneous fat involving edema or inflammation, bowel disease, arthritis, myopathy, heart disease, cancer, acute or chronic inflammatory demyelinating polyneuropathy, systemic inflammatory response syndrome, idiopathic inflammatory demyelinating disease, multiple sclerosis, progressive inflammatory neuropathy, immune-mediated inflammatory disease, idiopathic inflammatory myopathies, inflammatory vascular disease, acute inflammatory demyelinating polyneuropathy, Guillain Bane syndrome, prostatitis, allergies, systemic inflammation related to obesity or metabolic syndrome, autoimmune mediated inflammation, diabetes mellitus type 1, autoimmune peripheral neuropathy, atopic dermatitis, Becets Disease, systemic vascular inflammation, chronic recurrent multifocal osteomyelitis, inflammation related to tissue injury subsequent to cancer treatment, osteomyelitis, coeliac disease, dermatomyositis, eczema, neruodermatitis, gastritis, glomerulonephritis, and combinations thereof;
1. the ailment is a post-surgical outcome caused by a member of the group consisting of: tissue or organ transplant rejection, a xenograft, failure of implanted synthetic materials, an inflammatory rejection response, abdominal fistula, abdominal herniation, tendon repair, ligament repair, cartilage repair, meniscus repair, joint repair or replacement, repair of tissue-to-tissue interfaces, herniation of skin or abdominal wall, implantation of artificial dentures or teeth, pain, swelling, and combinations thereof;
J. the ailment is an inflammatory condition of skin selected from the group consisting of: dermatitis, atopic dermatitis, contact dermatitis, pain and swelling caused by treatment for infections of the skin, scabies, eczema, cellulitis, allergic reactions and inflammation caused by poisonous plants, an inflammatory response to allergens, an inflammatory reaction to insect stings and bites, vasospasm, a urticaria-class condition, an angioedema-class condition, Raynaud's phenomenon, an auto inflammatory syndrome, chronic blistering, inflammation or edema of mucous membranes, a pruritic skin condition, striae distensae, gravidarum, lichen planus, mucinoses, psoriasis, and combinations thereof;
k. the ailment is an inflammatory condition, pain, or edema of a musculoskeletal system or craniofacial structures selected from the group consisting of: fasciitis, plantar fasciitis, fibromyalgia, myasthenia gravis, a non-immunosuppressive responsive myasthenic syndrome, periodontitis, and combinations thereof;
1. the ailment is a musculoskeletal condition selected from the group consisting of: low bone density, damage from bone scaffolding, calcium buildup in arthritic areas due to injuries, treatment of a degenerative disease of a musculoskeletal system, and combinations thereof, wherein the degenerative disease of the musculoskeletal system is juvenile idiopathic and rheumatic arthritis, adult rheumatic arthritis and osteoarthritis, polymyositis, chondromalacia, relapsing polychondritis, rheumatoid arthritis, hiatal hernia, a systemic inflammatory disorder, synovitis, or scleritis;
m. the ailment is selected from the group consisting of: tinnitus, hearing loss related to inflammation around or damage to auditory nerves, damaged optic nerve or retina, damaged cranial or facial nerves, facial paralysis, pars planitis, intermediate uveitis, vitritis, macular edema, cystoid macular edema, neuromyelitis optics, Wegener's granulomatosis, and combinations thereof,
n. the ailment is selected from the group consisting of: hamstrings, sprains, pulled muscles, strains, bruises, and other sports related and occupation physical injuries;
o. the ailment is cancer, wherein the plurality of magnetic trapezoidal-wave pulses are configured to disrupt cancer cells, reduce a patency or growth rate of cancer cells, and reduce neoplastic tissue genesis; or
p. combinations thereof.
